(19) United States
(12) Patent Application Publication
(10) Pub. No.: US 2010/0222334 A1
(43) Pub. Date: Sep. 2, 2010

(54) Pharmaceutical Formulation of Valsartan

(76) Inventors: Wayne Talamonti, Kunkletown, PA (US); Robert Frank Wagner, Hillsborough, NJ (US); Hong Wen, Westfield, NJ (US)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/681,657

(22) PCT Filed: Oct. 7, 2008
(86) PCT No.: PCT/US08/79009

§ 371 Date: Apr. 5, 2010

Related U.S. Application Data
(60) Provisional application No. 60/978,531, filed on Oct. 9, 2007.

(51) Int. Cl. A61K 31/41; A61K 31/5415; A61P 9/12; A61P 9/00; A61P 9/10; A61P 13/12; A61P 25/00

(52) U.S. Cl. 514/224.2; 514/381

(57) ABSTRACT

The present invention relates to a pharmaceutical composition in a form of suspension for oral administration comprising valsartan or its pharmaceutically acceptable salts and at least one or two or more of the components selected from glycerol or syrup or the mixture thereof, a preservative, a buffer system and a suspending/stabilizing agent. The present invention further relates to the therapeutic uses of the pharmaceutical composition.

### FIELD OF THE INVENTION

The present invention relates to a pharmaceutical composition in a form of valsartan suspension forms and the therapeutic uses thereof.

### BACKGROUND OF THE INVENTION

Valsartan, i.e. (S)—N-(1-carboxy-2-methylprop-1-yl)-N-pentanoyl-N-[2′-(1H-tetrazol-5-yl)biphenyl-4-ylmethyl]amine, is a weakly acidic drug compound. The structure, preparation and formulation of valsartan is described for instance in U.S. Pat. No. 5,399,578, U.S. Pat. No. 6,294,197, WO 97/49394, WO 00/38676 and WO 01/97805, the contents of which are hereby incorporated into the present application by reference. Valsartan is an angiotensin II receptor antagonist and is effective and well tolerated in the treatment of congestive heart failure and reduction of blood pressure. Its combination with hydrochlorothiazide (HCTZ) is also known for the treatment of hypertension.

Valsartan is currently marketed as an immediate release tablet formulation (Diovan® containing valsartan 40 mg, 80 mg, 160 mg or 320 mg. Valsartan shows a low bioavailability (around 30%) and relatively high inter- and intra-subject variability when administered in this form. Unfortunately, the tablet is difficult for children or senior adults to swallow.

### SUMMARY OF THE INVENTION

In one aspect, the present invention relates to a pharmaceutical composition in a form of suspension comprising valsartan or its pharmaceutically acceptable salts in a liquid medium for oral administration. In one embodiment, the pharmaceutical composition comprises a therapeutically effective amount of valsartan or its pharmaceutically salts in a liquid medium comprising at least one or two or three or more of the following components including glycerol or syrup or the mixture thereof, a preservative, a buffer system and a suspending/stabilizing agent, etc.

In one embodiment, valsartan is employed in an amount ranging from about 0.1 mg/ml to about 16 mg/ml, or from about 0.25 mg/ml to about 8 mg/ml, or from about 1 mg/ml to about 4 mg/ml, or about 0.25 mg/ml, or about 0.5 mg/ml, or about 1 mg/ml, or about 2 mg/ml, or about 4 mg/ml, or about 5 mg/ml, or about 8 mg/ml, or about 10 mg/ml, or about 12 mg/ml, or about 14 mg/ml or about 16 mg/ml. The amount of valsartan noted above refers to the amount of free valsartan present in a given suspension form.

In one embodiment, the valsartan oral suspension of the present invention has a pH around 4.0. Also in another embodiment, the valsartan suspension of the present invention has a pH range of 3.0 to 5.0. Examples of the buffer system useful in the present invention include but are not limited to, citrate buffers, phosphate buffers, or any other suitable buffer known in the art. Preferably the buffer system include sodium citrate, potassium citrate, sodium bicarbonate, potassium bicarbonate, sodium dihydrogen phosphate and potassium dihydrogen phosphate, etc. The concentration of the buffer system in the final suspension varies according to factors such as the strength of the buffer system and the pH/pH ranges required for the suspension. In one embodiment, the concentration is within the range of 0.005 to 0.5 w/v % in the final suspension.

In addition to the aforementioned components, the valsartan oral suspension form can also optionally contain other excipients commonly found in pharmaceutical compositions such as alternative solvents, taste-masking agents, antioxidants, fillers, acidifiers, enzyme inhibitors and other components as described in Handbook of Pharmaceutical Excipients, Rowe et al., Eds., 4^{th} Edition, Pharmaceutical Press (2003), which is hereby incorporated by reference.

In another aspect, the present invention provides a process for preparing the valsartan suspension. The process comprises steps of bringing valsartan or its pharmaceutically acceptable salts thereof into mixture with the components including glycerol or syrup or the mixture thereof, a preservative, a buffer system and a suspending/stabilizing agent, etc., in a liquid medium. In general, the valsartan oral suspension is prepared by uniformly and intimately mixing these various components in the liquid medium.

Yet another embodiment of the present invention is directed to a method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache or chronic heart failure, comprising administering a therapeutically effective amount of the pharmaceutical composition in a form of valsartan suspension to a subject in need of such treatment. In a preferred embodiment, the suspension is orally administered to the subject.

The valsartan suspension of the present invention exhibits surprisingly advantageous properties when administered orally, e.g., in terms of consistency and high level of bioavailability obtained in standard bioavailability trials. Pharmacokinetic parameters, e.g., drug substance absorption and measured, e.g., as blood levels, also become surprisingly more predictable and problems in administration with erratic absorption may be eliminated or reduced. In addition, the function of the valsartan suspension upon oral administration may also reduce variability in inter- and intra-patient dose response.

In another aspect of the present invention, the valsartan suspension can be used in combination with a second therapeutic agent. For example, a valsartan suspension of the present invention can further comprise an antihypertensive agent selected from diuretics, calcium channel blockers (CCB), beta-blockers and ACE inhibitors, etc.

### DETAILED DESCRIPTION

In one aspect, the present invention relates to a pharmaceutical composition in a form of suspension comprising valsartan or its pharmaceutically acceptable salts in a liquid medium for oral administration. In one embodiment, the pharmaceutical composition comprises a therapeutically effective amount of valsartan or its pharmaceutically salts in a liquid medium comprising at least one or two or three or more of the following components including glycerol or syrup or the mixture thereof, a preservative, a buffer system and a suspending/stabilizing agent, etc.

In one embodiment, valsartan is employed in an amount ranging from about 0.1 mg/ml to about 16 mg/ml, or from about 0.25 mg/ml to about 8 mg/ml, or from about 1 mg/ml to about 4 mg/ml, or about 0.25 mg/ml, or about 0.5 mg/ml, or about 1 mg/ml, or about 2 mg/ml, or about 4 mg/ml, or about 5 mg/ml, or about 8 mg/ml, or about 10 mg/ml, or about 12 mg/ml, or about 14 mg/ml or about 16 mg/ml. The amount of valsartan noted above refers to the amount of free valsartan present in a given suspension form.

The pharmaceutical composition comprising the valsartan suspension form of the presentation can also include a preservative to prevent the growth of micro-organisms such as bacteria, yeasts and fungi, etc. Suitable preservatives could be selected from any one or more of: chlorhexidine; methyl paraben; propyl paraben; butyl paraben and their salts; diazolidinyl urea (Germall II.RTM); quaternary compounds, eg benzalkonium chloride and cetylpyridinium chloride, phenyl ethyl alcohol and the like. The concentration of preservatives may range from about 0.01% to about 0.5% (w/v)

In a suspension form, it is desirable to have a particular pH and/or to be maintained within a specific pH range. For example, valsartan is observed to have pH-dependent solubility. Certain pH or pH ranges for a drug substance such as valsartan would ensure optimal absorption or bioavailability after administration. In order to control the pH, a suitable buffer system can be used. In addition, the buffer system should have sufficient capacity to maintain the desired pH range. Preferably the valsartan oral suspension of the present invention has a pH around 4.0. Also preferably the valsartan suspension of the present invention has a pH range of 3.0 to 5.0. Examples of the buffer system useful in the present invention include but are not limited to, citrate buffers, phosphate buffers, or any other suitable buffer known in the art. Preferably the buffer system include sodium citrate, potassium citrate, sodium bicarbonate, potassium bicarbonate, sodium dihydrogen phosphate and potassium dihydrogen phosphate, etc. The concentration of the buffer system in the final suspension varies according to factors such as the strength of the buffer system and the pH/pH ranges required for the suspension. In one embodiment, the concentration is within the range of 0.005 to 0.5 w/v % in the final suspension.

The pharmaceutical composition comprising the valsartan suspension of the present invention can also include a suspending/stabilizing agent to prevent settling of the active material. Over time the settling could lead to caking of the active to the inside walls of the product pack, leading to difficulties with redispersion and accurate dispensing. Suitable stabilising agents include but are not limited to, the polysaccharide stabilizers such as xanthan, guar and tragacanth gums as well as the cellulose derivatives HPMC (hydroxypropyl methylcellulose), methyl cellulose and Avicel RC-591 (microcrystalline cellulose/sodium carboxymethyl cellulose). In another embodiment, polyvinylpyrrolidone (PVP) can also be used as a stabilizing agent.

In addition to the aforementioned components, the valsartan oral suspension form can also optionally contain other excipients commonly found in pharmaceutical compositions such as alternative solvents, taste-masking agents, antioxidants, fillers, acidifiers, enzyme inhibitors and other components as described in Handbook of Pharmaceutical Excipients, Rowe et al., Eds., 4^{th} Edition, Pharmaceutical Press (2003), which is hereby incorporated by reference.

Valsartan is slightly soluble in water but more soluble in alcohols. Accordingly, adding the alternative solvents can help increase valsartan's solubility in the suspension, and consequently the absorption and bioavailability inside the body of a subject. Preferably the alternative solvents include methanol, ethanol or propylene glycol and the like.

The pharmaceutical composition comprising the valsartan suspension form can also optionally include one or more taste-making agents. A taste-masking agent can be a sweetener, a flavoring agent or a combination thereof. The sweetener can be a sugar or a sugar substitute selected from lactose, mannitol, sucrose, glucose, or a mixture of the above. The sugar is most preferably sucrose. The taste-masking agents typically provide up to about 0.1% or 5% by weight of the total pharmaceutical composition.

A flavoring agent herein is a substance capable of enhancing taste or aroma of a composition. Suitable natural or synthetic flavoring agents can be selected from standard reference books, for example Fenaroli's Handbook of Flavor Ingredients, 3rd edition (1995). Non-limiting examples of suitable natural flavors, some of which can readily be simulated with synthetic agents or combinations thereof, include almond, anise, apple, apricot, bergamot, blackberry, blackcurrant, blueberry, cacao, caramel, cherry, cinnamon, clove, coffee, coriander, cranberry, cumin, dill, eucalyptus, fennel, fig, ginger, grape, grapefruit, guava, hop, lemon, licorice, lime, malt, mandarin, molasses, nutmeg, orange, peach, pear, peppermint, pineapple, raspberry, rose, spearmint, strawberry, tangerine, tea, vanilla, wintergreen, etc. Also useful, particularly where the composition is intended primarily for pediatric use, is tutti-frutti or bubblegum flavor, a compounded flavoring agent based on fruit flavors. Presently preferred flavoring agents include anise, cinnamon, cacao, orange, peppermint, cherry (in particular wild cherry), grape, bubblegum and vanilla. Wild cherry is particularly preferred. Flavoring agents can be used singly or in combinations of two or more. Typically the flavoring agent, or an oil or essence comprising the flavoring agent, if present is at a concentration in the composition of about 0.1 to about 5 mg/ml, preferably about 0.2 to about 3 mg/ml, and most preferably about 0.5 to about 2 mg/ml.

Examples of antioxidants include, but are not limited to, ascorbic acid and its derivatives, tocopherol and its derivatives, butyl hydroxyl anisole and butyl hydroxyl toluene. Vitamin E as α-tocopherol is particularly useful.

Examples of fillers include, but are not limited to, microcrystalline cellulose, silicone dioxide, starch and its derivatives, lactose, dicalcium phosphate and mannitol.

Examples of acidifiers include, but are not limited to, citric acid, succinic acid, fumaric acid, Ascorbic acid, phosphric acid, capric acid, oleic acid, glutamic acid and hydroxypropyl methyl cellulose acetate succinate, cellulose acetate phthalate, cellulose acetate trimellitate, hydroxypropyl methyl cellulose phthalate, carboxymethyl ethyl cellulose and carbomer.

For purposes of interpreting this specification, the following definitions will apply and whenever appropriate. In addition, terms used in the singular in the specification will also include the plural and vice versa.

As used herein, the term “pharmaceutically acceptable” is meant a material that is not biologically or otherwise undesirable, i.e., the material may be administered to an individual along with the selected active agent without causing any undesirable biological effects or interacting in a deleterious manner with any of the other components of the pharmaceutical composition in which it is contained.

As used herein, the term “pharmaceutically acceptable salts” refers to salts that retain the biological effectiveness and properties of the compounds of this invention and, which are not biologically or otherwise undesirable. In many cases, the compounds of the present invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in Remington's Pharmaceutical Sciences, 20th ed., Mack Publishing Company, Easton, Pa., (1985), which is herein incorporated by reference.

The term “therapeutically effective amount” of a compound of the present invention refers to an amount of the compound of the present invention that will elicit the biological or medical response of a subject, or ameliorate symptoms, slow or delay disease progression, or prevent a disease, etc.

As used herein, the term “subject” or “individual” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “a disorder” or “a disease” refers to any derangement or abnormality of function; a morbid physical or mental state. See Dorland's Illustrated Medical Dictionary, (W.B. Saunders Co. 27th ed. 1988).

As used herein, the term “treating” or “treatment” of any disease or disorder refers in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder. Also as used herein, the term “treating” or “treatment” also refers to preventing the recurrence of a disease, disorder or condition or of one or more symptoms associated with such disease, disorder or condition.

As used herein, the term “a,” “an,” “the” and similar terms used in the context of the present invention (especially in the context of the claims) are to be construed to cover both the singular and plural unless otherwise indicated herein or clearly contradicted by the context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

As used herein, the terms “drug,” “active agent” and “therapeutic agent” are used interchangeably herein to refer to a chemical material or compound which, when administered to an organism (human or animal), induces a desired pharmacologic effect. Included are analogs and derivatives (including salts, esters, prodrugs, and the like) of those compounds or classes of compounds specifically mentioned which also induce the desired pharmacologic effect.

In another aspect, the present invention provides a process for preparing the valsartan suspension. The process comprises steps of bringing valsartan or its pharmaceutically acceptable salts thereof into mixture with the components including glycerol or syrup or the mixture thereof, a preservative, a buffer system and a suspending/stabilizing agent, etc., in a liquid medium. In general, the valsartan oral suspension is prepared by uniformly and intimately mixing these various components in the liquid medium. For example, the components such as glycerol or syrup or the mixture thereof, a preservative, a buffer system and a suspending/stabilizing agent, etc., can be dissolved in water to form the aqueous solution, then valsartan can be then dispersed in the aqueous solution to form the suspension. Additionally, alternative solvents, taste-masking agents, antioxidants, fillers, acidifiers, enzyme inhibitors and the like can optionally be added into the suspension.

The resulted valsartan suspension can be in a liquid volume of 10 ml to 30 ml, preferably 20 ml, and the valsartan can be in an amount ranging from about 0.1 mg/ml to about 16 mg/ml, or from about 0.25 mg/ml to about 8 mg/ml, or from about 1 mg/ml to about 4 mg/ml, or about 0.25 mg/ml, or about 0.5 mg/ml, or about 1 mg/ml, or about 2 mg/ml, or about 4 mg/ml, or about 5 mg/ml, or about 8 mg/ml, or about 10 mg/ml, or about 12 mg/ml, or about 14 mg/ml or about 16 mg/ml. The amount of valsartan noted above refers to the amount of free valsartan present in a given suspension form. Such unit dosage forms are suitable for administration 1-5 times daily depending upon the particular purpose of therapy, the phase of therapy and the like.

Yet another embodiment of the present invention is directed to a method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache or chronic heart failure, comprising administering a therapeutically effective amount of the pharmaceutical composition in a form of valsartan suspension to a subject in need of such treatment. In a preferred embodiment, the suspension is orally administered to the subject.

In another aspect of the present invention there is provided a use of a pharmaceutical composition in a form of valsartan suspension for the manufacture of a medicament for the treatment of hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache or chronic heart failure.

The valsartan suspension of the present invention exhibits surprisingly advantageous properties when administered orally, e.g., in terms of consistency and high level of bioavailability obtained in standard bioavailability trials. Pharmacokinetic parameters, e.g., drug substance absorption and measured, e.g., as blood levels, also become surprisingly more predictable and problems in administration with erratic absorption may be eliminated or reduced. In addition, the function of the valsartan suspension upon oral administration may also reduce variability in inter- and intra-patient dose response.

In another aspect of the present invention, the valsartan suspension can be used in combination with a second therapeutic agent. For example, a valsartan suspension of the present invention can further comprise an antihypertensive agent selected from diuretics, calcium channel blockers (CCB), beta-blockers and ACE inhibitors, etc.

A diuretic is, for example, a thiazide derivative selected from the group consisting of chlorothiazide, hydrochlorothiazide, methylclothiazide, and chlorothalidone. The most preferred is hydrochlorothiazide.

A useful CCB is preferably a DHP representative selected from the group consisting of amlodipine, felodipine, ryosidine, isradipine, lacidipine, nicardipine, nifedipine, niguldipine, niludipine, nimodipine, nisoldipine, nitrendipine, and nivaldipine, and is preferably a non-DHP representative selected from the group consisting of flunarizine, prenylamine, diltiazem, fendiline, gallopamil, mibefradil, anipamil, tiapamil and verapamil, and in each case, a pharmaceutically acceptable salt thereof.

A beta-adrenergic receptor blocker includes esmolol especially the hydrochloride thereof, acebutolol, alprenolol, amosulalol, arotinolol, atenolol, befunolol, betaxolol, bevantolol, bisoprolol, bopindolol, bucumolol, bufetolol, bufuralol, bunitrolol, buprandolol, butiridine hydrochloride, butofilolol, carazolol, carteolol, carvedilol, celiprolol, cetamolol, cloranolol, dilevalol, epanolol, indenolol, labetalol, levobunolol, mepindolol, metipranolol, metoprolol, moprolol, nadolol, nadoxolol, nebivalol, nipradilol, oxprenolol, perbutolol, pindolol, practolol, pronethalol, propranolol, sotalol, sufinalol, talindol, tertatolol, tilisolol, timolol, toliprolol, and xibenolol, or in each case, a pharmaceutically acceptable salt thereof.

An ACE inhibitor is selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, in each case, a pharmaceutically acceptable salt thereof.

Specific embodiments of the invention will now be demonstrated by reference to the following examples. It should be understood that these examples are disclosed solely by way of illustrating the invention and should not be taken in any way to limit the scope of the present invention.

### EXAMPLES

### Example 1

### Preparation of Valsartan Suspension

### Materials

DIOVAN® 80 mg film-coated tablets (commercial stock).
Placebo DIOVAN® tablets, basis number 3761921.006 (material no. 850527).
Ora-Sweet SF™ Syrup Vehicle, NDC no. 0574-0302-16 (sugar-free, alcohol-free aqueous based vehicle containing glycerin, sorbitol, sodium saccharin, xantham gum, and flavoring; buffered with citric acid and sodium citrate; methylparaben (0.03%), propylparaben (0.008%) and potassium sorbate (0.1%) as preservatives) (Paddock Laboratories, Inc.).
Ora-Plus® Oral Suspending Vehicle, NDC no. 0574-0303-16 (suspending agent containing microcrystalline cellulose, carboxymethylcellulose sodium, xanthan gum, carrageenan, citric acid and sodium phosphate as buffers; simethicone as antifoaming agent; methylparaben and potassium sorbate as preservatives) (Paddock Laboratories, Inc.).
180 ml glass amber bottle for oral liquids (sourced from Huningue) item no. 30437, PN no. 9400120.
Closure for 180 ml glass amber bottle, child-resistant, SK TRIL KS 28 (sourced from Huningue) item no. 32798, PN no. 9400062.
ExactaMed® Oral Syringe Dispenser, 10 ml standard oral dispenser with bottle adapter in mini-grip bag (Baxa Limited, sourced from Huningue), item no. 33022, PN no. 9400100.

### Preparation Process

For extemporaneous preparation of the 640 mg/160 ml original suspension formulation, the instructions remain the same. Eight DIOVAN® 80 mg tablets are added to the dispensing bottle. Using a graduated cylinder, 80 ml of Ora-Plus™ Suspending Vehicle is added to the bottle and subsequently shaken for a minimum of 2 minutes. The suspension has a stand-time of at least 1 hour. After the standing time, the suspension is shaken for a minimum of 1 minute. Following shaking, using a graduated cylinder, 80 ml of Ora-Sweet™ SF Syrup Vehicle is added to the bottle. The final extemporaneous suspension is shaken for 10 seconds to disperse the ingredients.

For extemporaneous preparation of the placebo oral suspension, 8 Placebo DIOVAN® tablets is used to prepare 160 ml, using the same preparation procedure as described above for the 640 mg/160 ml suspension.

For extemporaneous preparation of 40 mg/160 ml, 80 mg/160 ml, 160 mg/160 ml and 320 mg/160 ml (2 mg/ml) oral suspensions, the required quantity of the 640 mg/160 ml dosage form is used and subsequently diluted to a final volume of 160 ml using the placebo oral suspension. The required volumes for each oral suspension are listed above in Table 1.

For all strengths, a 10 ml oral dispensing syringe is used to measure the required volume of 640 mg/160 ml oral suspension, except for 320 mg/160 ml suspension. For the 320 mg/160 ml oral suspension, a graduated cylinder is used to measure the required volume of 640 mg/160 ml suspension. For all strengths, the required volume of 0 mg/160 ml is measured using a graduated cylinder.

To prepare 40 mg/160 ml, 80 mg/160 ml, 160 mg/160 ml and 320 mg/160 ml oral suspensions, the required volume of 640 mg/160 ml oral suspension is dispensed into an empty, glass amber bottle. Using a graduated cylinder, the required volume of 0 mg/160 ml is added to the same bottle. The resultant suspension is shaken for 10 seconds to disperse the ingredients.

TABLE 1
Valsartan suspension
Strength

| | 0 mg/160 ml | 40 mg/160 ml | 80 mg/160 ml | 160 mg/160 mL | 320 mg/160 mL | 640 mg/160 mL |
|---|---|---|---|---|---|---|
| | (0 mg/20 ml) | (5 mg/20 ml) | (10 mg/20 ml) | (20 mg/20 mL) | (40 mg/20 mL) | (80 mg/20 mL) |
| Volume of | 0 ml | 10 ml | 20 ml | 40 ml | 80 ml | 160 ml |
| 640 mg/160 ml | | | | | | |
| required | | | | | | |
| Volume of | 160 ml | 150 ml | 140 ml | 120 ml | 80 ml | 0 ml |
| 0 mg/160 ml | | | | | | |
| required | | | | | | |
| Oral bottle | 180 ml | 180 ml | 180 ml | 180 ml | 180 ml | 180 ml |
| (glass | | | | | | |
| amber) | | | | | | |

### Example 2

### Bioavailability Studies

A suspension formulation of valsartan is used in clinical studies to characterize pharmacokinetics in 1-16 yr old children and efficacy in 1-5 yr old children. Since the suspension formulation is a significant change to the currently marketed valsartan tablet, this study is conducted to determine the bioavailability of 20 mL of 4 mg/mL valsartan extemporaneous oral suspension relative to one 80 mg valsartan tablet (Diovan®). The study is conducted in healthy subjects using a two-way, two period crossover study design with a seven-day inter-dose washout period. Pharmacokinetic samples are collected for up to 24 hours postdose. A total of 32 healthy male subjects are enrolled in the study and 30 subjects complete both treatment periods and the data are included in the pharmacokinetic analysis. The pharmacokinetic and statistical results are summarized in Table 2. The results of this study have showed that valsartan is absorbed quickly with a mean Tₘₐₓ of 1.6 hr and 2.7 hr when administered as a suspension and tablet, respectively. The rate of valsartan absorption as determined as Cₘₐₓ is higher with suspension formulation compared to the commercial tablet by 1.93-fold. The extent of valsartan absorption estimated as AUC₀₋ₜ, and AUC_{0-inf} is also higher with suspension formulation by 1.58- and 1.56-fold, respectively. These results are similar to earlier observations wherein valsartan solution results in higher bioavailability compared to valsartan capsules

TABLE 2
Summary pharmacokinetic parameters of valsartan following single oral
administration of 20 mL of 4 mg/mL valsartan suspension and 80 mg
valsartan marketed Tablet

| | | | Ratio of | |
|---|---|---|---|---|
| Pharmacokinetic | | | geometric | 90% CI for ratio of |
| parameter | Suspension | Marketed Tablet | means* | geometric means* |
| AUC₀₋ₜ (μg · h/mL) | 16.13 ± 6.3 | 10.77 ± 4.93 | 1.58 | 1.37-1.82 |
| AUC_{0-∞} (μg · h/mL) | 16.77 ± 6.61 | 11.63 ± 5.0 | 1.56 | 1.36-1.78 |
| Cₘₐₓ (μg/mL) | 3.13 ± 1.17 | 1.76 ± 0.96 | 1.93 | 1.59-2.32 |
| tₘₐₓ (h)^{#} | 1.6 (1.0, 4.0) | 2.7 (1.5, 6.0) | | |

*Log-transformed parameters are analyzed;
^{#}mean, min, and max are presented

A 10 mg valsartan tablet is used to determine the dose response and safety in pediatric patients of age 6-16 years. In order to determine the relative bioavailability, an open-label, single dose, two-period, crossover study is conducted in 24 healthy subjects. Subjects received either 4×10 mg valsartan tablets (Clinical Service Forms) or a commercial 40 mg valsartan tablet in a randomized manner and all subjects completed both treatment periods. Plasma concentrations of valsartan are monitored up to 48 h post dose in both treatments. The pharmacokinetic results including statistical analysis are summarized in Table 3. The study results have showed that valsartan was absorbed quickly with median Tmax of 2.5 to 3.0 hr in both treatments. The rate of valsartan absorption measured as Cmaxwas 8% higher with 4×10 mg tablets compared to 40 mg commercial tablet. Also, the extent of absorption measured as AUC₀₋ₜ and AUC_{0-inf} are about 12% higher with 4×10 mg valsartan tablets. Since the intersubject variability (CV %) was in the range of 24-40% for the Cₘₐₓ and AUC_{τ} the observed differences in Cₘₐₓand AUC of valsartan are not considered significant.

TABLE 3
Summary analysis of valsartan pharmacokinetic parameters following
single oral administration of 4 × 10 mg valsartan pediatric tablets and 40 mg
valsartan commercial tablet

| Pharmacokinetic | | 40 mg | Ratio of geometric | 90% CI for ratio of |
|---|---|---|---|---|
| parameter | 4 × 10 mg tablet | marketed tablet | means* | geometric means* |
| AUC₀₋ₜ (μg · h/mL) | 11.62 ± 4.45 | 9.93 ± 3.1 | 1.12 | 0.96-1.31 |
| AUC_{0-∞} (μg · h/mL) | 11.99 ± 4.75 | 10.23 ± 3.31 | 1.12 | 0.97-1.31 |
| Cₘₐₓ (μg/mL) | 1.8 ± 0.6 | 1.5 ± 0.4 | 1.08 | 0.90-1.29 |
| Tₘₐₓ (h)^{#} | 2.5 (1.5, 6.0) | 3.0 (1.0, 4.0) | | |

*Log-transformed parameters were analyzed;
^{#}median, min, and max are presented

A new 80 mg valsartan pediatric tablet is developed for the use in clinical trials to determine safety and efficacy in pediatric patients and preserve blinding. Therefore, the bioavailability of the new 80 mg valsartan pediatric formulation is characterized relative to the 80 mg valsartan commercial tablet. The study is conducted in 24 healthy subjects using an open-label, single-dose, two period, randomized, crossover study design. All 24 subjects completed the study and are included in the pharmacokinetic data analysis. Plasma concentrations of valsartan are monitored up to 48 hours post dose. The pharmacokinetic results have including statistical analysis were summarized in Table 4. The study results indicated that following a single-dose administration, valsartan is absorbed rapidly with both formulations with a similar Tₘₐₓ of ˜3.0 hours. The 90% Cls for both rate (Cₘₐₓ) and extent (AUC₀₋ₜ and AUC_{0-∞}) of valsartan were slightly above the upper boundary of the interval with a point estimate of 1.11 and 1.09, respectively. The slight increase in rate and extent of absorption of valsartan are not relevant as the variability of valsartan PK is in the range of 30-50% in the same study.

TABLE 4
Summary analysis of valsartan pharmacokinetic parameters following
single oral administration of 80 mg valsartan pediatric tablet and 80 mg
valsartan commercial tablet.

| | | | Ratio of | |
|---|---|---|---|---|
| Pharmacokinetic | CSF - 80 mg | FMI - 80 mg tablet | geometric | 90% CI for ratio of |
| parameter | tablet (Test) | (Reference) | means* | geometric means* |
| AUC₀₋ₜ (μg · h/mL) | 17.04 ± 8.3 (48.5) | 15.11 ± 5.4 (35.5) | 1.09 | 0.94-1.28 |
| AUC_{0-∞} (μg · h/mL) | 17.46 ± 8.4 (48.1) | 15.70 ± 5.4 (34.6) | 1.08 | 0.93-1.26 |
| Cₘₐₓ (μg/mL) | 2.6 ± 1.3 (50.3) | 2.3 ± 0.7 (30.1) | 1.06 | 0.86-1.31 |
| tₘₐₓ^{†} (h) | 3.17 (1.5, 6.0) | 3.3 (1.5, 8.0) | | |

*Log-transformed parameters were analyzed;
^{†}Median (minimum, maximum) values are presented.

### Abbreviations

AUC₀₋ₜ Area under the serum concentration-time curve from time zero to time t, using the log-linear trapezoidal rule. Concentrations below the LOQ are set to zero and therefore excluded from the calculation. Actual sample collection times are used. Where ₀₋ₜ is shown as this denotes the AUC under a dosing interval

AUC_{0-∞} Area under the serum concentration-time curve from time zero to infinity. For extrapolation to infinity Cₗₐₛₜ/λ_{z} is used, where Cₗₐₛₜ is the estimated concentration at the last sample time point above LOQ from linear regression of the terminal elimination phase

Cₘₐₓ Maximum serum concentration after a single dose

CL_{CR} Creatinine clearance

CLₚ Plasma or serum clearance, calculated as Dose/AUC_{0-∞} after an intravenous dose

F Fraction of the dose systemically available

FDA Food and Drug Administration

i.v. Intravenous

LOQ Limit of quantification

LLOQ Lower limit of quantification

p.o. Oral

R Accumulation index, calculated as AUC_{τ} steady-state/AUC_{τ} single dose

t_{1/2}, λ_{z} Apparent terminal elimination half-life (t_{1/2}) or rate constant (λ_{z}), calculated from at least three consecutive data points and with an r² value≧0.75

tₘₐₓ Time to the maximum observed plasma concentration

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

1. A pharmaceutical composition in a form of suspension for oral administration comprising:
(a) valsartan or its pharmaceutically acceptable salts; and
(b) at least one or two or more of the components selected from glycerol or syrup or the mixture thereof, a preservative, a buffer system and a suspending/stabilizing agent.

2. The pharmaceutical composition of claim 1, wherein valsartan is employed in an amount ranging from about 0.1 mg/ml to about 16 mg/ml.

3. The pharmaceutical composition of claim 2, wherein valsartan is employed in an amount ranging from about 0.25 mg/ml to about 8 mg/ml.

4. The pharmaceutical composition of claim 1, wherein said buffer system maintains the pH of the composition in the range of about 3.0 to about 5.0.

5. The pharmaceutical composition of claim 1, wherein said buffer system maintains the pH of 4.0.

6. The pharmaceutical composition of claim 1, wherein said buffer system is selected from sodium citrate, potassium citrate, sodium bicarbonate, potassium bicarbonate, sodium dihydrogen phosphate and potassium dihydrogen phosphate.

7. The pharmaceutical composition of claim 1, wherein said preservative is about 0.01% to about 0.5% (w/v).

8. The pharmaceutical composition of claim 1, further comprising an alternative solvent, a taste-masking agent, a filler, an acidifier, an antioxidant or a mixture thereof.

9. The pharmaceutical composition of claim 1, further comprising a second anti-hypertensive agent.

10. The pharmaceutical composition of claim 9, the anti-hypertensive agent is selected from diuretics, calcium channel blockers (CCB), beta-blockers and ACE inhibitors.

11. The pharmaceutical composition of claim 9, the anti-hypertensive agent is hydrochlorothiazide.

12. A method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure comprising administering a therapeutically effective amount of a pharmaceutical composition of claim 1 to a subject in need of such treatment.

13. A method of a pharmaceutical composition in a form of suspension for oral administration for the manufacture of a medicament for the treatment of hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive: dysfunction, headache or chronic heart failure.
